(19) United States
(12) Patent Application Publication
(10) Pub. No.: US 2004/0208931 A1
(43) Pub. Date: Oct. 21, 2004

(54) Fast dissolving films for oral administration of drugs

(76) Inventors: David R. Friend, Princeton, NJ (US); Aaron W. Levine, Lawrenceville, NJ (US); Kerrie L. Ziegler, Hammonton, NJ (US); Emmanuel Manna, Monmouth Junction, NJ (US)

Correspondence Address: William Squire, Esq., c/o Carella, Byrne, Bain,Gilfillan,, Cecchi,Stewart &Olstein, 5 Becker Farm Road, Roseland, NJ, 07068, US

(21) Appl. No.: 10/744,479

(22) Filed: Dec. 23, 2003

Related U.S. Application Data
(60) Provisional application No. 60/437,137, filed on Dec. 30, 2002.

(51) Int. Cl. A61K 9/24

(52) U.S. Cl. 424/471

(57) ABSTRACT

A dosage unit comprises a substrate comprising a first polymer; a deposit, including an active ingredient; and a cover layer comprising a second polymer, wherein the cover layer covers the deposit and is joined to the first surface of the substrate by a bond that encircles the deposit and wherein at least one of the first and second polymers is a graft co-polymer. The dosage unit wherein said first and second polymers may be the same, and also the graft co-polymer may be a polyvinyl alcohol-polyethylene glycol graft co-polymer. Also disclosed is a dosage unit wherein the deposit is formed on the substrate by electrostatic dry drug deposition. The dosage unit may also include a polymer that is a graft co-polymer; and an active ingredient, and the graft co-polymer may be polyvinyl alcohol-polyethylene glycol.

### BACKGROUND OF THE INVENTION

Solid pharmaceutical dosages traditionally have included orally-administered, solid shaped articles such as capsules, tablets and other unit dosage forms, each form containing a pharmaceutically or biologically active ingredient and at least one additional “excipient” ingredient. The excipient, which is intended to be a therapeutically inert and non-toxic carrier, may function, for example, as a diluent, binder, lubricant, disintegrant, stabilizer, buffer or preservative. These standard oral dosage forms are designed for short residence time in the mouth, that is, they are intended to be swallowed whole or chewed since absorption of the agent from these dosage forms occurs in the gastrointestinal tract.

Various active pharmaceutical ingredients, when admixed with excipients, have exhibited problems relating to chemical stability. Some of the most widely used “inert” excipients may, in fact, be quite reactive in their own right. Drugs can interact with excipients via a number of mechanisms, resulting in chemical instability and degradation.

Another limitation of the traditional solid formulations is the difficulty associated with swallowing the solid dosage forms for geriatric and pediatric patients.

What is needed is a dosage formulation which is easily administered and where necessary, avoids the instability caused by interaction of the active ingredient with excipients.

### SUMMARY OF THE INVENTION

In one aspect, the invention relates to a dosage unit comprising a fast-dissolving film comprising a polymer that is a graft co-polymer; and an active ingredient. The active ingredient may be incorporated into the film prior to casting of the film or may be deposited according to a number of known methods onto a pre-formed film layer.

In a related aspect, the invention relates to a dosage unit comprising a substrate comprising a first polymer, a deposit, including an active ingredient and a cover layer comprising a second polymer, wherein the cover layer covers the deposit and is joined to a first surface of the substrate by a bond that encircles the deposit and wherein at least one of the first and second polymers is a graft co-polymer. In one embodiment of the dosage unit, the substrate and cover layer comprise the same polymer.

In a related aspect, the invention relates to a dosage unit comprising a polyvinyl alcohol-polyethylene glycol graft co-polymer. In one embodiment, the active ingredient is deposited upon the substrate by electrostatic dry drug deposition.

### DETAILED DESCRIPTION OF THE INVENTION

All patents, applications, publications, or other references that are listed herein are hereby incorporated by reference. In the description that follows, certain conventions will be followed as regards the usage of terminology:

The term “active ingredient” refers to a therapeutically or pharmaceutically active substance.

The term “dry drug deposition” refers to a method of depositing a material without using a liquid vehicle.

The terms “electrostatic dry deposition” and “electro-attractive dry deposition” refer to methods that use an electrostatically-charged surface or an electromagnetic field to dry deposit charged powder.

The term “graft co-polymer” refers to a copolymer in which chains of a first polymer made of monomer B are grafted onto a second polymer chain of monomer A. A preferred graft co-polymer for use in the present invention is a co-polymer consisting of chains of polyvinyl alcohol grafted onto a polyethylene glycol backbone.

The present invention contemplates a dosage form comprising a rapidly dissolving film which when administered to a mucosal surface releases a pharmaceutically active agent. In the following description, specific reference may be made to the oral cavity by way of example. However, it is not intended to limit the scope of the invention to the oral cavity.

In one embodiment, the dosage form of the present invention comprises an edible film in which the active ingredient is incorporated during preparation of the film, that is, before the film is cast. A dosage form comprising the grafted co-polymer film of the present invention has the advantage of quicker disintegration and dissolution times as well as improved characteristics, for example, mouth feel, when compared to conventional film-forming polymers.

In another embodiment, the dosage form comprises a substrate and cover layer each comprising a substantially planar, flexible film or sheet. In some embodiments, one of either substrate or cover layer includes an array of semi-spherical bubbles, concavities, blisters or depressions arranged in columns and rows.

The film comprises a fast-dissolving, water-soluble graft co-polymer and, optionally, one or more pharmaceutically acceptable ingredients, for example, a permeation enhancer. The dosage unit formulation of the present invention further comprises an active ingredient, either alone or, optionally, in combination with another active ingredient, or other excipients, including surfactants, sweetening agents and the like. One advantage of the present invention, however, is the ability to prepare a solid dosage formulation which avoids the instability caused by the interaction of certain active ingredients with excipients.

A preferred polymer for use in producing the film of the dosage form of the present invention is a graft co-polymer; a preferred co-polymer being one of polyvinyl alcohol (PVA) and polyethylene glycol (PEG). The PVA-PEG graft co-polymer is available as Kollicoat® IR (BASF, Mount Olive, N.J.). The PVA-PEG graft co-polymer consists of 75% polyvinyl alcohol units and 25% polyethylene glycol units with PEG providing the backbone of the branched co-polymer, with the PVA forming the branches. PVA-PEG is very readily soluble in water and has been used mainly for the production of instant-release coatings for tablets.

Methods for manufacturing the film of the dosage unit of the invention include the solvent casting method described in Z. W. Wicks, F. Jones and S. P. Pappas, Organic Coatings: Science and Technology, Vol. 1; Film Formation, Components and Appearance. Wiley, NY 1992. In one embodiment of the invention, the solvent casting method employs a polymer that is completely dissolved or dispersed in water or in a water alcohol solution under mixing to form a homogeneous formulation. Solutions of Kollicoat® IR with concentrations of up to 40% can be prepared in water and aqueous systems, for example, weak acids or bases. Solutions of up to 25% can be prepared in a 1:1 ethanol-water mixture.

The homogeneous mixture with a solid content of 5-40% and more preferably 5-25% is degassed and coated onto a smooth surface, for example, the non-siliconized side of a polyester film, and dried under aeration at a temperature between 30-80° C. The dry film formed by this process is a glossy stand alone, self-supporting, non-tacky and flexible film.

If the active ingredient has not already been incorporated into the film during its preparation, the film is now ready for deposition of the active ingredient. The dry film is then cut into a suitable shape and surface area for active agent delivery at the preferred site. For example, the cast film can be die-cut into different shapes and sizes using a rotary die. The film may be cut into a size that contains for example, a single dosage unit. For example, a single dosage unit may include a film size with surface area of 5 cm² that contains a dosage of active agent in the range of 20-250 mg. The size of the film may be varied according to the dosage required. The dosage contained in each square centimeter is selected according to the active agent. Films are ultimately packaged into a single pouch package, multi-unit blister card or multiple unit dispensers (U.S. Pat. No. 6,394,306 and U.S. application Ser. No. 10/122,808).

In one embodiment of the invention, the active agent is deposited onto a substrate layer using an electrostatic deposition process such as the one described in U.S. Pat. No. 6,319,541, U.S. Pat. No. 5,699,649, U.S. Pat. No. 5,960,609, and WO 006/64592. In that process, a cloud or stream of charged particles of the active ingredient is exposed to, or directed towards a substrate at the surface of which substrate a pattern of opposite charges has been established. In this fashion, a measured dosage of the active ingredient can be adhered to a substrate without the need for additional carriers, binders or the like.

Other suitable means of electrostatic deposition are described in, for example, U.S. Pat. Nos. 5,714,007, 5,846,595 and 6,074,688, the disclosures of which are incorporated by reference herein in their entirety. In addition to the electrostatic powder cloud deposition method, the active ingredient may be coated onto the substrate in the form of a solution or a suspension of finely divided medicament, for example, a colloidal suspension.

Following deposition of the active ingredient, substrate and cover layer are attached to one another via bonds or welds that are near to and encircle the deposited material. Bonding can be effected, for example, via heat or ultrasonic welding or via suitable adhesives.

The dosage unit may be prepared for use by selecting a film that is capable of delivering an effective dose and administering the film to the patient by placing it on a mucosal surface such as the oral mucosa where it dissolves in the body fluid, for example, saliva and is swallowed in liquid form or absorbed via the mucosal tissue. Absorbtion through the mucosal tissue can be facilitated by the incorporation of a permeation enhancer into the film.

The rate at which the active ingredient is released from the dosage unit is determined by a number of factors. These factors include: the concentration of the active agent, solubility of the agent at the mucosal surface and the dimensions of the unit dosage form, including film thickness. The thickness of the film is a factor in determining the rate of dissolution. Generally, a thick film will dissolve more slowly than a thin film. A thick film, however, may be desirable for its greater holding capacity for active agents that are required in higher dosages. The film used in producing the dosage form of the present invention has the unexpected advantage of rapid disintegration and dissolution times even at increased thicknesses of film. Preferred thicknesses for films of the dosage unit of the present invention are 2.0 to 8.0 mils (1 mil=0.001 in.), and more preferred 3 to 5 mils.

The dosage unit of the invention may be used as a vehicle for delivering a wide range of active agents, such as ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha₂-adrenergic agonists, analgesics, antipyretics and anti-inflammatory agents, androgens, anesthetics, antiaddictive agents, antiandrogens, antiarrhythmic agents, antiasthmatic agents, anticholinergic agents, anficholinesterase agents, anficoagulents, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatlet agents, antiprogestins, antithyroid agents, antitussives, antiviral agents, a typical antidepressants, azaspirodecanediones, barbituates, benzodiazepines, benozthiadiazides, beta-adrenergic agonists, beta-adrenergic antagonists, selective beta₁-adrenergic antagonists, selective beta₂-adrenergic agonists, bile salts, agents affecting volume and composition of body fluids, butyrophenones, agents affecting calcification, calcium channel blockers, cardiovascular drugs, catecholamines and sympathomimietic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, hematopoietic agents, histamines, histamine antagonists, 5-hydroxytryptamine antagonists, drugs for the treatment of hyperlipoproteinemia, hypnotics and sedatives, immunosupressive agents, laxatives, methylxanthines, monamine oxidase inhibitors, neuromuscular blocking agents, organic nitrates, opiod analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. Although extensive, this list is not intended to be comprehensive.

1. A dosage unit comprising:
(a) a substrate comprising a first polymer;
(b) a deposit, including an active ingredient; and
(c) a cover layer comprising a second polymer, wherein said cover layer covers the deposit and is joined to said first surface of said substrate by a bond that encircles the deposit and
wherein at least one of said first and second polymers is a graft co-polymer.

2. The dosage unit of claim 1, wherein said first and second polymers are the same.

3. The dosage unit of claim 1, wherein said graft co-polymer is a polyvinyl alcohol-polyethylene glycol graft co-polymer.

4. The dosage unit of claim 1, wherein said deposit is made on said substrate by electrostatic dry drug deposition.

5. A dosage unit comprising:
(a) a fast-dissolving film comprising a polymer that is a graft co-polymer; and
(b) an active ingredient.

6. The dosage unit of claim 5, wherein said graft co-polymer is a polyvinyl alcohol-polyethylene glycol graft co-polymer.
